(19) United States
(12) Patent Application Publication
(10) Pub. No.: US 2022/0313758 A1
(43) Pub. Date: Oct. 6, 2022

(54) LACTOBACILLUS COMPOSITIONS AND USES THEREOF

(71) Applicant: Probi AB, Lund (SE)

(72) Inventor: Michael Svensson, Umeå (SE)

(21) Appl. No.: 17/620,604

(22) PCT Filed: Jun. 17, 2020
(86) PCT No.: PCT/EP2020/066722

§ 371 Date: Dec. 17, 2021

(30) Foreign Application Priority Data
Jun. 18, 2019 (GB) 1908706.3

(51) Int. Cl. A61K 35/747

(52) U.S. Cl. CPC A61K 35/747; C12R 2001/25

(57) ABSTRACT

The invention relates to the use of at least one strain of Lactobacillus plantarum to increase vigour in a human.

### TECHNICAL FIELD OF THE INVENTION

The present invention relates to the use of at least one strain of Lactobacillus plantarum to increase vigour in a human.

By “vigour” in the context of the present invention is meant mental energy, enthusiasm and determination.

Skilled persons will appreciate that vigour is a psychological state that can be assessed by a variety of methods. For example, the Profile of Mood States (POMS) is a validated psychological rating scale used to assess transient, distinct mood states. (McNair, D.; Lorr, M.; Doppleman, L. (1971). POMS Manual for the Profile of Mood States. San Diego, Calif.: Educational and Industrial Testing Service). The POMS test measures six different dimensions of mood swings over a period of time. These include: Tension or Anxiety, Anger or Hostility, Vigour or Activity, Fatigue or Inertia, Depression or Dejection, Confusion or Bewilderment.

Composed of 65 questions, those taking the test are asked to give a self-report for each question on how well they do or do not relate. A five-point Likert scale ranging from “not at all” to “extremely” is administered by experimenters to patients to assess their mood states. With an extensive number of questions, the data that is collected from the test can provide the participants with data that is reliable and consistent. Advantages of using this assessment include the simplicity of administration and ease of participant understanding.

POMS can be administered and measured through written or online forums. For example, an online questionnaire and assessment tool is provided by MACKENZIE, B. (2001) Profile of Mood States (POMS) [WWW] Available from: https://www.brianmac.co.uk/poms.htm [Accessed May 31, 2019], which is incorporated herein by reference and discussed in more detail with reference to the Table below.

### STATEMENT OF THE INVENTION

The invention provides the use of at least one strain of Lactobacillus plantarum to increase vigour in a human as defined in the accompanying claims.

The invention also provides at least one strain of Lactobacillus plantarum for use in a method of increasing vigour in a human compared to their vigour without treatment, comprising treatment by administering to a human at least one strain of Lactobacillus plantarum as defined in the accompanying claims.

The invention further provides a method of increasing vigour in a human compared to their vigour without treatment, the method comprising treatment by administering to a human at least one strain of Lactobacillus plantarum as defined in the accompanying claims.

Subject/Patient Group

Preferably, the human to be treated with at least one strain of Lactobacillus plantarum in accordance with the invention is in need of increased vigour.

It will be appreciated that a person in need of increased vigour may be suffering from decreased vigour. For example, they may suffer from lethargy. By “lethargy”, we include the meaning of a pathological state of sleepiness and/or unresponsiveness and inactivity. Hence the term is intended to cover physical and/or mental inertness; and a lack of energy and enthusiasm.

Lethargy is a condition that may be induced by a variety of factors, including one or more of disease, illness, injury and exposure to drugs, including exposure to agents used in medical treatment regimens, such as chemotherapy.

Morgan & Johnson (MORGAN, W. P. and JOHNSON, R. W. (1978) “Personality characteristics of successful and unsuccessful oarsmen.” International Journal of Sport Psychology, 9, p. 119-133) found that plotting the mood state results of elite performers prior to competition exhibited the FIG. 1 graph. This graph, with a raised peak for Vigour, was termed the “Iceberg” profile, identifying successful performers.

Accordingly, a person in need of increased vigour may not actually be suffering from a decrease in vigour, but they may wish to increase or boost vigour relative to their normal state. For example, this could be before and/or after an event or experience associated with a decrease in vigour, such as physical exercise and/or a medical procedure or treatment. In particular, a person in need of increased vigour may be a person about to undertake physical exertion, especially in an athletic event or competition.

Preferably, the human to be treated according to the invention has iron deficiency. By “iron deficiency” we include the meaning that the person has a mean plasma ferritin level of below 30 μg/litre, but without iron deficiency anemia. Most preferably, the human to be treated has a mean plasma ferritin level of 15-30μg/L without iron deficiency anemia.

Preferably, the human to be treated is an athlete. By “athlete” we include the meaning that, on average, the person does at least five hours vigorous exercise per week.

Preferably, the human to be treated according to the invention is female, with a female athlete being particularly preferred. The female may also be pre-, peri-, or post-menopausal. Preferably, the female is premenopausal.

In particular, the at least one strain according to the first aspect of the invention is suitable to be used in humans (men and/or women), including adolescents (aged 12-16 years old), adults aged 16-40 years old, and adults older than 40, 50, 60, 70 and 80 years old.

The human to be treated may be an individual that does not have chronic stress.

By “chronic stress” we include the meaning of a score of 3.75 or greater in the Shirom-Melamed Burnout Questionnaire (SMBQ) (Grossi et al, 2003, J Psychosomatic Res 55: 309-316). Hence, the human to be treated may have chronic stress indicated by a score of 3.75 or greater in the SMBQ. Alternatively, the human to be treated may not have chronic stress, indicated by a score of less than 3.75 in the SMBQ.

Preferably the human subject to be treated according to the invention does not have depression. By “depression” we include the meaning of a score of 45 or greater in the Zung Self-Rating Depression Scale (Zung, 1965, Arch Gen Psychiatry, 12:63-70, the entire contents of which are incorporated herein by reference).

Lactobacillus Strains

The Lactobacillus strains for use according to the invention and its methods may be viable, inactivated or dead. Preferably, the strains are viable. Most preferably, the strains for use according to the invention are probiotic.

Probiotic bacteria are defined as “live microorganisms that, when administered in adequate amounts, confer a health benefit on the host” (Hill et al, Nat Rev Gastroenterol Hepatol, 2014, 11(8):506-514).

By “probiotic strain” we include the meaning of a strain of bacteria which when administered in adequate amounts confer a health benefit on the host. Typically, the administration of said at least one probiotic strain will alter the composition of the gut microbiota.

The strain for use according to the invention is at least one strain of Lactobacillus plantarum.

Preferably, the at least one strain of Lactobacillus plantarum is chosen from Lactobacillus plantarum DSM 15312 (HEAL 9™), Lactobacillus plantarum DSM 15313 (HEAL 19™), Lactobacillus plantarum DSM 15316 (HEAL 99 ™), Lactobacillus plantarum DSM 6595 (299™), Lactobacillus plantarum DSM 9843 (299v®), Lactobacillus plantarum DSM 32131 (GOS42™), Lactobacillus plantarum DSM 17852 (LB3e™), and Lactobacillus plantarum DSM 17853 (LB7c™).

Lactobacillus plantarum DSM 15312 (HEAL 9™), Lactobacillus plantarum DSM 15313 (HEAL 19™), and Lactobacillus plantarum DSM 15316 (HEAL 99™), were deposited on 27 Nov. 2002 at DSMZ-DEUTSCHE SAMMLUNG VON MIKROORGANISMEN UND ZELLKULTUREN GmbH, Mascheroder Weg 1b, D-38124 Braunschweig, Germany, by Probi AB.

Lactobacillus plantarum DSM 6595 (299™) was deposited on 2 Jul. 1991 at DSM-DEUTSCHE SAMMLUNG VON MIKROORGANISMEN UND ZELLKULTUREN GmbH, Mascheroder Weg 1 B, D-3300 Braunschweig, Germany, in the name of Probi (i.e. Probi AB).

Lactobacillus plantarum DSM 9843 (299v®) was deposited on 16 Mar. 1995 at DSM-DEUTSCHE SAMMLUNG VON MIKROORGANISMEN UND ZELLKULTUREN GmbH, Mascheroder Weg 1b, D-38124 Braunschweig, Germany, by Probi AB.

Lactobacillus plantarum DSM 32131 (GOS42™) was deposited on 2 Sep. 2015 at Leibniz Institute DSMZ-German Collection of Microorganisms and Cell Cultures, lnhoffenstr. 7 B, D-38124 Braunschweig, Germany by Probi AB.

Lactobacillus plantarum DSM 17852 (LB3e™) and Lactobacillus plantarum DSM 17853 (LB7c™) were deposited on 6 Jan. 2006 at DSMZ-Deutsche Sammlung von Mikroorganismen and Zellkulturen GmbH, Mascheroder Weg 1b, D-38124 Braunschweig, Germany, by Probac AB. All rights and duties in connection with microorganism deposits DSM 17852 and DSM 17853 were subsequently given to and accepted by Probi AB, who is now the depositor of the DSM 17852 and DSM 17853 strains.

Preferably, the at least one strain of Lactobacillus plantarum is able to adhere to the intestinal epithelium and persist in the intestine. Particular strains of this species comprise a mannose-specific adhesin (Adlerberth et al, 1996, Appl Environ Microbiol, 62(7):2244-2251, the entire contents of which are incorporated herein by reference), including Lactobacillus plantarum DSM 15312 (HEAL 9™), Lactobacillus plantarum DSM 15316 (HEAL 99™) Lactobacillus plantarum DSM 9843 (299v®) and Lactobacillus plantarum DSM 6595 (299v®).

Most preferably, the at least one probiotic strain of Lactobacillus plantarum is Lactobacillus plantarum DSM 9843 (299v®)

Compositions

The at least one Lactobacillus plantarum strain according to the first aspect of the invention may be present in a composition comprising at least one pharmaceutically and/or nutritionally acceptable carrier, diluent or excipient material. The composition may be as a solid or liquid formulation, and hence the at least one carrier may be a solid or a liquid, or may comprise both at least one solid component and at least one liquid component.

Examples of a suitable liquid carrier include water, milk, coconut water, fruit drinks and juices, milk substitutes (soya drink, oat drink, nut and other plant-based drinks), sparkling beverages, oil formulations including one or more of a nut or vegetable oil, such as coconut, rapeseed, olive, palm, corn/maize; glycerin, propylene glycol; and aqueous solvents.

Examples of a suitable solid carrier or excipient include maltodextrin, inulin, a cellulose such as microcrystalline cellulose (MCC), hydroxypropylmethylcellulose (HPMC) or hydroxy-propylcellulose (HPC), sugar alcohols, high molecular weight polyethylene glycols, lactose, sodium citrate, calcium carbonate, dibasic calcium phosphate and glycine, disintegrants such as starch (preferably corn, potato, tapioca or other vegetable starch), sodium starch glycollate, croscarmellose sodium and certain complex silicates, and granulation binders such as polyvinylpyrrolidone, sucrose, gelatin and acacia. Additionally, lubricating agents such as magnesium stearate, stearic acid, glyceryl behenate and talc may be included.

The carrier may be selected from a pharmaceutically acceptable carrier, excipient, or diluent; and a nutritionally acceptable i.e. food-grade carrier, excipient, or diluent material. For example, the carrier material may be a food.

Examples of suitable “pharmaceutically acceptable” carriers, excipients and diluents include those well known to a skilled person in the art, for example those given in Remington: The Science and Practice of Pharmacy, 19^{th} ed., vol. 1 & 2 (ed. Gennaro, 1995, Mack Publishing Company).

By “nutritionally acceptable” or “food-grade” we include carriers, ingredients and excipients that meet the ‘generally recognized as safe’ (GRAS) criteria.

By “food” we include any substance for consumption to provide nutritional benefit or support for an organism. Examples of suitable food carriers include beverages (e.g.

juices), dairy products (e.g. yoghurts, cheese, ice creams, infant formula and spreads such as margarine), dairy-alternative products (e.g. soy, nut or other plant-based drinks, yoghurts and spreads), cereal-based products (e.g. breads, biscuits, breakfast cereals, pasta and dry food bars such as health bars), and baby food (e.g. pureed fruit and/or vegetable).

The composition according to the invention may be a dry, non-fermented composition, a fermented composition, or a dry, fermented composition. Fermentation in this context particularly includes lactic acid fermentation by lactic acid bacteria in anaerobic conditions. In the case of a dry, non-fermented composition, substantially no fermentation takes place before ingestion by a subject, and so fermentation only takes place in the gastrointestinal tract after ingestion of the composition by a subject.

Hence, in some embodiments according to the invention, the composition is in the form of a food wherein the food is a cereal-based product, a dairy product, a juice drink, or a fermented food.

Examples of fermented foods include fermented milk products (such as yoghurt, kefir or lassi), fermented dairy-free milk alternatives (such as coconut milk kefir), fermented cereal-based products (such as oats, oatmeal, maize, sorghum, wheat), fermented vegetables (such as sauerkraut, kimchi, or pickles), fermented legumes or soybeans (such as natto or tempeh) and fermented tea (such as kombucha).

In use, the at least one strain or the composition comprising the at least one strain according to the invention may be mixed with a liquid or solid carrier before administration. For example, one may mix the strain or the composition thereof with a carrier comprising one or more liquids chosen from water, milk, coconut water, fruit drinks and juices, milk substitutes (soya drink, oat drink, nut and other plant-based drinks), sparkling beverages or some other aqueous solvent or drink prior to intake. Similarly, the at least one strain or the composition thereof may be mixed with a carrier consisting of one or more foods. Suitable food carriers include oatmeal carrier, barley carrier, fermented or non-fermented dairy products such as yoghurts, ice creams, milkshakes, fruit juices, beverages, soups, breads, biscuits, pasta, breakfast cereals, dry food bars including health bars, plant-based foods such as soy products, spreads, baby food, infant nutrition, infant formula, breast milk replacements from birth.

Preferably, the formulation is a unit dosage containing a daily dose or unit, daily sub-dose or an appropriate fraction thereof, of the composition comprising the probiotic strains.

The composition according to the invention may be a dietary supplement. By “dietary supplement” we include the meaning of a manufactured product intended to supplement the diet when taken by mouth, e.g. as a pill, capsule, tablet, or liquid. Dietary supplements may contain substances that are essential to life and/or those that have not been confirmed as being essential to life but may have a beneficial biological effect. When the composition according to the invention is in the form of a dietary supplement the carrier(s) to be added include those well known to a skilled person in the art, for example those given in Remington: The Science and Practice of Pharmacy, 19^{th} ed., vol. 1 & 2 (ed. Gennaro, 1995, Mack Publishing Company). Any other ingredients that are normally used in dietary supplements are known to a skilled person and may also be added conventionally together with the strain.

In a most preferred embodiment the composition of the invention is supplemented with iron, preferably an iron salt and most preferably the iron salt is ferrous fumarate.

The composition according to the invention may be provided in the form of a solution, suspension, emulsion, tablet, granule, powder, capsule, lozenge, chewing gum, or suppository.

In a preferred embodiment, the composition according to the invention is provided in the form of a capsule.

Hence, in an especially preferred embodiment of the invention the composition is provided as a capsule containing 1×10¹⁰ CFU Lactobacillus plantarum DSM 9843 (299v®) and 20 mg iron (e.g. as ferrous fumarate).

Dose and Methods of Treatment

Conveniently, the at least one strain is administered at a dose of from about 1×10⁶ to about 1×10¹⁴ CFU/dose, preferably from about 1×10⁸ to about 1×10¹² CFU/dose, more preferably from about 1×10⁹ to about 1×10¹¹ CFU/dose, and most preferably about 1×10¹⁰ CFU/dose.

If the at least one strain consists of more than one strain, such amounts represent the total CFU/dose of the combination of strains.

Preferably, the daily dose of at least one Lactobacillus strain is administered for at least four weeks, more preferably for at least eight weeks; and most preferably for at least twelve weeks.

The composition according to the invention can be administered orally, buccally or sublingually in the form of tablets, capsules, powders, ovules, elixirs, solutions or suspensions, which may contain flavouring or colouring agents, for immediate-, delayed- or controlled-release applications. The composition may be administered in the form of a powdered composition such as a fast-melt microbial composition, for example those described in WO 2017/060477, or in Probi's UK Patent Application 1708932.7 or Probi's publication WO 2018/224509 relating to Probi® Fast Melt technology, the entire contents of all three of which are incorporated herein by reference. Where the powder is not in a fast-melt microbial composition, it may be suitable for being added to a food (e.g. yoghurt) or drink (e.g. water or milk) before ingestion.

Where the composition is in the form of a powder, it would typically be filled in a sealed container, which provides an oxygen and moisture barrier in order to protect and maintain the viability of the bacteria in the composition. Hence, where the composition is in the form of a powder, preferably the composition is packaged in sealed aluminium foil sticks, where each stick comprises one dose of the composition, i.e. one dose of the bacteria. Non-limiting examples of suitable containers include a stick, bag, pouch or capsule. In a preferred embodiment, the container is an aluminium foil or a polyethylene stick, which is typically sealed by welding. The stick is typically configured for easy tear opening. The stick may have a tear notch.

The composition according to the invention may be formulated as a controlled-release solid dosage form, for example any of those described in WO 03/026687 and U.S. Pat. Nos. 8,007,777 and 8,540,980, the entire contents of which are incorporated herein by reference. The composition may be formulated as a layered dosage form, for example Probi's BIO-tract® technology including any of the layered dosage forms described in WO 2016/003870, the entire contents of which are incorporated herein by reference.

A tablet may be made by compression or moulding, optionally with one or more accessory ingredients. Compressed tablets may be prepared by compressing in a suitable machine the at least one strain (e.g. freeze-dried) in a free-flowing form such as a powder or granules, optionally mixed with a binder (eg povidone, gelatin, hydroxypropylmethyl cellulose), lubricant, inert diluent, preservative, disintegrant (eg sodium starch glycolate, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide the desired release profile.

Administration according to the uses and methods of the invention may include administration once, twice, three, four, five, six or seven times a week. Preferably, administration takes place at least once daily.

Administration according to the uses and methods of the invention is preferably of a unit dosage of from about 1×10⁶ to about 1×10¹⁴ CFU/unit dose, preferably from about 1×10⁸ to about 1×10¹² CFU/unit dose, more preferably from about 1×10⁹ to about 1×101¹ CFU/unit dose, and most preferably about 1×10¹⁰ CFU/unit dose.

Preferably, the unit dose is administered once daily. Hence, administration according to the uses and methods of the invention preferably results in a daily dose of from about 1×10⁶ to about 1×10¹⁴ CFU/day, preferably from about 1×10⁸ to about 1×10¹² CFU/day, more preferably from about 1×10⁹ to about 1×10¹¹ CFU/day, and most preferably about 1×10¹⁰ CFU/day.

It will be appreciated that a preferable daily dose may also be achieved by administration of more than one sub-dose, for example, by a twice daily administration of a unit dose comprising half of the preferable daily dose. Hence, the preferred ranges for the effective dose may also represent the preferred daily dosage to be achieved in whatever number of unit doses is practical.

The subject may be instructed to consume a treatment effective dose of the at least one strain. or a composition thereof, in combination with water, another aqueous solvent or a food product, e.g. yoghurt.

### EXAMPLES

The invention will now be described in more detail by reference to the following Examples, Tables and Figures which embody aspects of the present invention.

### BRIEF DESCRIPTION OF THE FIGURES

FIG. 1: Graph plotting POMS test score against mood states, prior to competition, for successful and less successful performers from Morgan & Johnson (1978)-supra.

TABLE 1
online POMS Questionnaire (Mackenzie B. (2001) supra)

| | |
|---|---|
| Feeling | How I have felt** |

Friendly
Tense
Angry
Worn Out
Unhappy
Clear Headed
Lively
Confused
Sorry for things done
Shaky
Listless
Peeved
Considerate
Sad
Active
On Edge
Grouchy
Blue
Energetic
Panicky
Hopeless
Relaxed
Unworthy
Spiteful
Sympathetic
Uneasy
Restless
Unable to Concentrate
Fatigued
Helpful
Annoyed
Discouraged
Resentful
Nervous
Lonely
Miserable
Muddled
Cheerful
Bitter
Exhausted
Anxious
Ready to Fight
Good Natured
Gloomy
Desperate
Sluggish
Rebellious
Helpless
Weary
Bewildered
Alert
Deceived
Furious
Efficient
Trusting
Full of Pep
Bad Tempered
Worthless
Forgetful
Carefree
Terrified
Guilty
Vigorous
Uncertain about things
Bushed
**Selection from “Not at All”; “A Little”; “Moderately”; “Quite a Bit”; “Extremely”.

Assessment

A Total Mood Disturbance (TMD) score (possible range of −32 to 200) is calculated by adding scores for Tension (0-36), Depression (0-60), Anger (0-48), Fatigue (0-28) and Confusion (0-28) and then subtracting the Vigour score (0-32).

The scores in brackets (x-y) above indicate the possible score range with lower scores indicative of people with more stable mood profiles.

Normative Data

TERRY, P. (n.d.) Normative Values for the Profile of Mood States for Use with Athletic Samples, [WWW] Available from: https://eprints.usq.edu.au/4385/2/Terry_Lane_JASS_v12n1_Authors_version.pdf [Accessed Jun. 18, 2019]) provides POMS norms for an athletic sample (n=2086) grouped by level of competition (International standard athletes, club level athletes and recreational athletes).

| Group | Tension | Depression | Anger | Vigour | Fatigue | Confusion |
|---|---|---|---|---|---|---|
| International | 5.66 | 4.38 | 6.24 | 18.51 | 5.37 | 4.00 |
| Club | 9.62 | 8.67 | 9.91 | 15.64 | 8.16 | 7.38 |
| Recreational | 6.00 | 3.11 | 3.60 | 17.78 | 6.37 | 4.84 |

Analysis

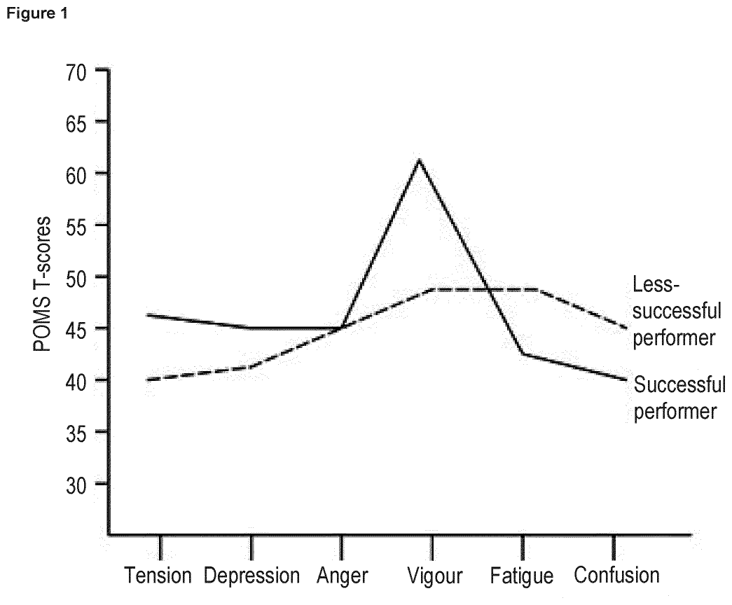

Analysis of the POMS result is by comparing it with the results of previous POMS tests. Morgan & Johnson (MORGAN, W. P. and JOHNSON, R. W. (1978) “Personality characteristics of successful and unsuccessful oarsmen.” International Journal of Sport Psychology, 9, p. 119-133) found that plotting the mood state results of elite performers prior to competition exhibited the FIG. 1 graph. This graph, with a raised peak for Vigour, was termed the “Iceberg” profile, identifying successful performers.

Exemplary Dosage Forms

In addition to the formulations referenced above (and incorporated herein by reference), the following examples illustrate pharmaceutical formulations and other formulations according to the invention.

Example A: Tablet

| Lactobacillus strain(s) | 1 × 10¹⁰ CFU | |
|---|---|---|
| Lactose | 200 | mg |
| Starch | 50 | mg |
| Polyvinylpyrrolidone | 5 | mg |
| Magnesium stearate | 4 | mg |

Tablets are prepared from the foregoing ingredients by wet granulation followed by compression.

Example B: Tablet Formulations

The following formulations A and B are prepared by wet granulation of the ingredients with a solution of povidone, followed by addition of magnesium stearate and compression.

Formulation A

| | | |
|---|---|---|
| (a) Lactobacillus strain(s) | 1 × 10¹⁰ CFU | 1 × 10¹⁰ CFU |

| | | | | |
|---|---|---|---|---|
| (b) Lactose B.P. | 210 | mg | 26 | mg |
| (c) Povidone B.P. | 15 | mg | 9 | mg |
| (d) Sodium Starch Glycolate | 20 | mg | 12 | mg |
| (e) Magnesium Stearate | 5 | mg | 3 | mg |

Formulation B

| | | |
|---|---|---|
| (a) Lactobacillus strain(s) | 1 × 10¹⁰ CFU | 1 × 10¹⁰ CFU |

| (b) Lactose | 150 | mg | — | |
|---|---|---|---|---|
| (c) Avicel PH 101 ® | 60 | mg | 26 | mg |
| (d) Povidone B.P. | 15 | mg | 9 | mg |
| (e) Sodium Starch Glycolate | 20 | mg | 12 | mg |
| (f) Magnesium Stearate | 5 | mg | 3 | mg |

Formulation C

| Lactobacillus strain(s) | 1 × 10¹⁰ CFU | |
|---|---|---|
| Lactose | 200 | mg |
| Starch | 50 | mg |
| Povidone | 5 | mg |
| Magnesium stearate | 4 | mg |

The following formulations, D and E, are prepared by direct compression of the admixed ingredients. The lactose used in formulation E is of the direction compression type.

Formulation D

| | |
|---|---|
| Lactobacillus strain(s) | 1 × 10¹⁰ CFU |
| Pregelatinised Starch NF15 | 150 mg |

Formulation E

| | |
|---|---|
| Lactobacillus strain(s) | 1 × 10¹⁰ CFU |
| Lactose | 150 mg |
| Avicel ® | 100 mg |

Formulation F (Controlled Release Formulation) The formulation is prepared by wet granulation of the ingredients (below) with a solution of povidone followed by the addition of magnesium stearate and compression.

| (a) Lactobacillus strain(s) | 1 × 10¹⁰ CFU | |
|---|---|---|
| (b) Hydroxypropylmethylcellulose | 112 | mg |
| (Methocel K4M Premium) ® | | |
| (c) Lactose B.P. | 53 | mg |
| (d) Povidone B.P.C. | 28 | mg |
| (e) Magnesium Stearate | 7 | mg |

Release takes place over a period of about 6-8 hours and was complete after 12 hours.

Example C: Capsule Formulations

Formulation A A capsule formulation is prepared by admixing the ingredients of Formulation D in Example B above and filling into a two-part hard gelatin capsule. Formulation B (infra) is prepared in a similar manner.

Formulation B

| (a) Lactobacillus strain(s) | 1 × 10¹⁰ CFU | |
|---|---|---|
| (b) Lactose B.P. | 143 | mg |
| (c) Sodium Starch Glycolate | 25 | mg |
| (d) Magnesium Stearate | 2 | mg |

Formulation C

| | |
|---|---|
| (a) Lactobacillus strain(s) | 1 × 10¹⁰ CFU |
| (b) Macrogol 4000 BP | 350 mg |

Capsules are prepared by melting the Macrogol 4000 BP, dispersing the probiotic strain(s) in the melt and filling the melt into a two-part hard gelatin capsule.

Formulation D (Controlled Release Capsule) The following controlled release capsule formulation is prepared by extruding ingredients a, b, and c using an extruder, followed by spheronisation of the extrudate and drying. The dried pellets are then coated with release-controlling membrane (d) and filled into a two-piece, hard gelatin capsule.

| (a) Lactobacillus strain(s) | 1 × 10¹⁰ CFU | |
|---|---|---|
| (b) Microcrystalline Cellulose | 125 | mg |
| (c) Lactose BP | 125 | mg |
| (d) Ethyl Cellulose | 13 | mg |

Example D: Powder Formulations

Formulation A (fast-melting microbial composition)

| (a) Lactobacillus strain(s) | 80 mg (preferably 1 × 10¹⁰ CFU) | |
|---|---|---|
| (b) Erythritol | 450 | mg |
| (c) Inulin | 227.5 | mg |
| (d) Xylitol | 227.5 | mg |
| (e) Lemon flavour | 10 | mg |
| (f) Silicon dioxide | 5 | mg |

Formulation B (fast-melting microbial composition)

| (a) Lactobacillus strain(s) | 80 mg (preferably 1 × 10¹⁰ CFU) | |
|---|---|---|
| (b) Erythritol | 425 | mg |
| (c) Inulin | 215 | mg |
| (d) Xylitol | 215 | mg |
| (e) Maltodextrin | 50 | mg |
| (f) Lemon flavour | 10 | mg |
| (g) Silicon dioxide | 5 | mg |

Study of Effect of Lactobacillus Treatment on Mood States in Humans

The study was a randomized, placebo controlled, double-blind, parallel group, single centre study. The Wilcoxon signed rank test was used when evaluating change over time within each group and Wilcoxon rank sum test was used when evaluating differences between groups. All reported p-values are two-sided and nominal, i.e. not adjusted for multiple testing.

As shown in Table 2, the evaluation of mood states using the POMS questionnaire revealed increased vigour after the 12 weeks of intake of the Lactobacillus strain as compared to control (3.5 vs. 0.1, p=0.0151, change from baseline to 12 weeks).

Study Design

The study was initiated by the screening of healthy males and females at 16 to 40 years of age involved in recreational training or elite sports with a minimum of 5 hours training per week (Visit 1). A total of 365 subjects were screened. Subjects displaying iron deficiency (low level of serum ferritin, i.e. <30 μg/L) but without anemia (low level of Hb) were eligible to be randomized into the intervention which included 12 weeks (Visits 2-5) of daily administration of the investigational Lactobacillus strain-containing product or control product which did not contain the Lactobacillus strain.

Investigational Product

Capsules containing freeze-dried Lactobacillus plantarum DSM 9843 (registered trade marks LP299v®; 299v®) at a concentration of 1×10¹⁰ CFU/capsule, 20 mg iron (ferrous-fumarate), maize starch (bulking agent), maltodextrin (bulking agent), cellulose derivatives (coating of minerals) and magnesium stearate (processing aid/anti-caking agent). The capsules themselves consisted of hydroxypropylmethyl cellulose and titanium dioxide.

The control product contained all but the Lactobacillus strain. The investigational and control products were packed in blister packs with 10 capsules per blister. One box contained four blisters (40 capsules) to provide adequate amount for a 4-week administration period.

Each subject filled out a POMS questionnaire related to perceived mood state.

Subject Disposition

In total, 365 subjects were included in the study. After the screening and initial blood analysis, 53 subjects fulfilled all inclusion criteria and none of the exclusion criteria and were randomized. During the study, 14 subjects were excluded or withdrew their consent.

Baseline Characteristics

No eligible male subjects were found in the screening process and therefore only female subjects were included in the study. The subjects in the Lp299v group trained for an average of 8.2±2.0 hours per week before the start of the study and the subjects in the control group trained for an average of 7.6±2.0 hours per week. There were no differences in average training, age, body weight or BMI between the groups at the start of the intervention.

RESULTS

Profile of Mood Score Reveals Increased Vigour Upon Treatment With Lactobacillus

The Profile of Mood States (POMS) questionnaire is a validated psychological test developed by McNair et al. (McNair, D.; Lorr, M.; Doppleman, L. (1971). POMS Manual for the Profile of Mood States. San Diego, Calif.: Educational and Industrial Testing Service). It was used to obtain a Total Mood Disturbance score and to analyse the Tension,

Depression, Anger, Vigour, Fatigue and Confusion sub-scores (see Table 2).

TABLE 2
Results from the POMS questionnaire at baseline and after 4, 8 and 12 weeks of supplementation.

| | | Baseline | | 4 weeks | | 8 weeks | | 12 weeks | | ΔV3-V2 | | |
|---|---|---|---|---|---|---|---|---|---|---|---|---|
| | | mean | std | mean | std | mean | std | mean | std | mean | Std (p-value) | |
| Total | Control | 124 | 22 | 120 | 20 | 122 | 34 | 118 | 29 | −3.2 | 23.2 | (0.9128) |
| POMS | | | | | | | | | | | | |
| | Lp299v | 129 | 30 | 126 | 30 | 120 | 25 | 120 | 27 | −3.3 | 19.4 | (0.9128) |
| Vigor | Control | 58 | 8 | 58 | 9 | 58 | 11 | 59 | 7 | −0.2 | 6.5 | (0.6232) |
| | Lp299v | 58 | 6 | 60 | 6 | 60 | 8 | 61 | 6 | 1.4 | 4.9 | (0.6232) |
| Tension | Control | 42 | 6 | 40 | 5 | 40 | 7 | 40 | 8 | −1.9 | 6.3 | (0.9213) |
| | Lp299v | 43 | 6 | 41 | 4 | 40 | 5 | 41 | 6 | −1.8 | 3.9 | (0.9213) |
| Depression | Control | 40 | 5 | 38 | 4 | 39 | 6 | 38 | 6 | −0.3 | 5.3 | (0.2662) |
| | Lp299v | 41 | 6 | 41 | 8 | 40 | 5 | 40 | 7 | 0.5 | 5.2 | (0.2662) |
| Anger | Control | 44 | 6 | 43 | 5 | 46 | 9 | 43 | 6 | −0.05 | 5.8 | (0.3720) |
| | Lp299v | 45 | 9 | 44 | 7 | 44 | 6 | 44 | 5 | −1.2 | 4.9 | (0.3720) |
| Fatigue | Control | 49 | 7 | 49 | 6 | 48 | 9 | 47 | 8 | 0.7 | 6.4 | (0.9659) |
| | Lp299v | 48 | 8 | 49 | 9 | 47 | 8 | 48 | 7 | 1 | 6.8 | (0.9659) |
| Confusion | Control | 41 | 5 | 41 | 5 | 40 | 7 | 40 | 6 | −0.6 | 5.0 | (0.4689) |
| | Lp299v | 43 | 5 | 42 | 6 | 40 | 4 | 42 | 6 | −1.4 | 4.4 | (0.4689) |

| | | | | | | | | | | | |
|---|---|---|---|---|---|---|---|---|---|---|---|
| n | Control | 23 | NA | 22 | NA | 15 | NA | 16 | NA | 22 | NA |
| | Lp299v | 17 | NA | 17 | NA | 16 | NA | 16 | NA | 16 | NA |

| | | ΔV4-V2 | | | ΔV5-V2 | | |
|---|---|---|---|---|---|---|---|
| | | mean | Std (p-value) | | mean | std (p-value) | |
| Total | Control | 4.3 | 32.3 | (0.2994) | 4.6 | 22.4 | (0.2199) |
| POMS | | | | | | | |
| | Lp299v | −11 | 30.2 | (0.2994) | −12.6 | 42.1 | (0.2199) |
| Vigor | Control | 1.2 | 9.8 | (0.6894) | 0.13 | 3.5 | (0.0151) |
| | Lp299v | 2.6 | 8.9 | (0.6894) | 3.5 | 6.3 | (0.0151) |
| Tension | Control | −0.9 | 5.8 | (0.5046) | −0.46 | 5.7 | (0.5179) |
| | Lp299v | −2.7 | 5.5 | (0.5046) | −3.2 | 8.3 | (0.5179) |
| Depression | Control | 0.7 | 0.67 | (0.9754) | 1.7 | 5.5 | (1.000) |
| | Lp299v | −1.2 | 6.1 | (0.9754) | −1.15 | 6.6 | (1.000) |
| Anger | Control | 4.1 | 6.9 | (0.0934) | 2.3 | 5.4 | (0.5012 |
| | Lp299v | −0.7 | 7.8 | (0.0934) | −1.6 | 11.4 | (0.5012) |
| Fatigue | Control | 1.3 | 8.3 | (0.6490) | 1.9 | 6.8 | (0.4116) |
| | Lp299v | −2 | 8.2 | (0.6490) | −2.6 | 10.9 | (0.4116) |
| Confusion | Control | 0.07 | 6.2 | (0.2602) | 0.5 | 4.6 | (0.5664) |
| | Lp299v | −3.3 | 5.7 | (0.2602) | −2.34 | 7.6 | (0.5664) |

| | | | | | |
|---|---|---|---|---|---|
| n | Control | 15 | NA | 16 | NA |
| | Lp299v | 15 | NA | 15 | NA |

V2 = Baseline
V3 = 4 weeks of supplementation
V4 = 8 weeks of supplementation
V5 = 12 weeks of supplementation

The results from the questionnaire show that the sub-score Vigour increased after 12 weeks of supplementation in the Lactobacillus treatment group (from a mean 58.0 at baseline to 61.43 at week 12, n=15-17, change=3.53, n=15, p=0.0436), but no differences were detected in the control group (from a mean 58.0 to 59.4, n=16-23, change=0.13, n=16, p=0.9814).

Also, the change from baseline to week 12 was significantly different compared to the control group (3.5 vs.0.13, p=0.0151).

No other differences between the groups could be detected neither for the total profile (from a mean 128.8 at baseline to 120.0 for Lp299v and 123.9 to 118 for control, change=−12.6 vs. 4.56, p=0.2199 nor for any of the other sub-scores (see Table 2).

Conclusions

The above study in humans shows that treatment by Lactobacillus plantarum administration resulted in an increase in vigour.

The listing or discussion of an apparently prior-published document in this specification should not necessarily be taken as an acknowledgement that the document is part of the state of the art or is common general knowledge.

1. At least one strain of Lactobacillus plantarum for use in a method of increasing vigour in a human compared to their vigour without treatment, comprising treatment by administering to a human at least one strain of Lactobacillus plantarum.

2. Use of at least one strain of Lactobacillus plantarum to increase vigour in a human.

3. A method of increasing vigour in a human compared to their vigour without treatment, the method comprising treatment by administering to a human at least one strain of Lactobacillus plantarum.

4. A use, at least one strain for use, or a method as claimed in any of claims 1 to 3, wherein the human to be treated is in need of increased vigour.

5. A use, at least one strain for use, or a method as claimed in claim 4, wherein the human to be treated has iron deficiency.

6. A use, at least one strain for use, or a method as claimed in any of claims 1 to 5, wherein the human is an athlete.

7. A use, at least one strain for use, or a method as claimed in any of claims 1 to 6, wherein the human is female, preferably a female athlete.

8. A use, at least one strain for use, or a method as claimed in claim 7, wherein the female is premenopausal.

9. A use, at least one strain for use, or a method according to any one of claims 1 to 8, wherein the at least one strain is administered at a daily dose of from about 1×10⁶ to about 1×10¹⁴ colony forming units (CFU).

10. A use, at least one strain for use, or a method according to any one of claims 1 to 9, wherein the daily dose of the at least one strain is from about 1×10⁹ to about 1×10¹¹ CFU.

11. A use, at least one strain for use, or a method according to claim 10, wherein the daily dose of the at least one strain is 1×10¹⁰ CFU.

12. A use, at least one strain for use, or a method according to any one of claims 1 to 11, wherein the at least one strain is administered at a daily dose in a single unit dosage form.

13. A use, at least one strain for use, or a method according to any one of claims 9 to 12, wherein the daily dose of at least one strain is administered for at least four weeks.

14. A use, at least one strain for use, or a method according to claim 13, wherein the daily dose of at least one strain is administered for at least eight weeks.

15. A use, at least one strain for use, or a method according to claim 14, wherein the daily dose of at least one strain is administered for at least twelve weeks.

16. A use, at least one strain for use, or a method according to any preceding claim, wherein the at least one strain of Lactobacillus plantarum is selected from Lactobacillus plantarum DSM 15312, Lactobacillus plantarum DSM 15313, Lactobacillus plantarum DSM 15316, Lactobacillus plantarum DSM 6595, Lactobacillus plantarum DSM 9843, Lactobacillus plantarum DSM 32131, Lactobacillus plantarum DSM 17852, and Lactobacillus plantarum DSM 17853.

17. A use, at least one strain for use, or a method according to any preceding claim, wherein the at least one strain of Lactobacillus plantarum is able to adhere to the intestinal epithelium and persist in the intestine.

18. A use, at least one strain for use, or a method according to any preceding claim, wherein the at least one strain of Lactobacillus plantarum comprises a mannose-specific adhesin.

19. A use, at least one strain for use, or a method according to any preceding claim, wherein the at least one strain of Lactobacillus plantarum is Lactobacillus plantarum DSM 9843 (299v®).

20. A use, at least one strain for use, or a method according to any preceding claim, wherein the at least one strain is administered in the form of a composition comprising at least one pharmaceutically and/or nutritionally acceptable carrier, excipient and/or diluent material.

21. A use, at least one strain for use, or a method according to claim 20, wherein the carrier material is a food.

22. A use, at least one strain for use, or a method according to claim 20 or 21, wherein the composition is provided in a form selected from a solution, suspension, emulsion, tablet, granule, powder, capsule, lozenge, chewing gum, or suppository.

23. A use, at least one strain for use, or a method according to claim 22, wherein the composition is provided in the form of capsule.

24. A use, at least one strain for use, or a method according to any of claims 20 to 23, wherein the composition comprises iron.

25. A use, at least one strain for use, or a method according to claim 24, wherein the iron is provided in the form of an iron salt.

26. A use, at least one strain for use, or a method according to claim 25, wherein the iron salt is ferrous fumarate.

27. A use, at least one strain for use, or a method according to any of claims 24 to 26 wherein the composition is provided in a capsule and contains Lactobacillus plantarum DSM 9843 (299v®) at a concentration of 1×10¹⁰ CFU/capsule and 20 mg iron, preferably ferrous-fumarate.

28. A use, at least one strain for use, or a method according to any preceding claim wherein increased vigour is measured using a Profile of Mood States (POMS) psychological rating scale.

29. A use, at least one strain for use, or a method as claimed in claim 30, wherein the increased vigour by treatment with at least one strain of Lactobacillus plantarum is not accompanied by improvements in one, two, three, four or all five of the other mood states of Tension, Depression, Anger, Fatigue, and Confusion, according to a POMS psychological rating scale.
